(19) United States
(12) Patent Application Publication
(10) Pub. No.: US 2019/0314301 A1
(43) Pub. Date: Oct. 17, 2019

(54) COMBINATIONS OF ADRENERGIC RECEPTOR AGONISTS FOR THE TREATMENT OF TYPE 2 DIABETES

(71) Applicant: ATROGI AB, Stockholm (SE)

(72) Inventors: Benjamin PELCMAN, Stockholm (SE); Tore BENGTSSON, Vaxholm (SE)

(73) Assignee: Atrogi AB, Stockholm (SE)

(21) Appl. No.: 16/317,009

(22) PCT Filed: Jul. 13, 2017
(86) PCT No.: PCT/GB2017/052066

§ 371 Date: Jan. 10, 2019

(30) Foreign Application Priority Data
Jul. 13, 2016 (GB) 1612165.9

(51) Int. Cl. A61K 31/137; A61K 31/426

(52) U.S. Cl. CPC A61K 31/137; A61K 31/426

(57) ABSTRACT

There is herein provided pharmaceutical formulations and kits-of-parts comprising a β₂-adrenergic receptor agonist, or a pharmaceutically acceptable salt thereof, and a β₃-adrenergic receptor agonist, or a pharmaceutically acceptable salt thereof. This combination of active agents finds particular utility in the treatment of type 2 diabetes.

### FIELD OF THE INVENTION

The present invention relates to methods for the treatment of type 2 diabetes, and to novel pharmaceutical formulations and kits-of-parts useful in the same. In particular, the invention relates to methods for the treatment of type 2 diabetes involving administering a combination of compounds capable of activating the β₂-adrenergic receptor and compounds capable of activating the β₃-adrenergic receptor.

### BACKGROUND OF THE INVENTION

The listing or discussion of an apparently prior-published document in this specification should not necessarily be taken as an acknowledgement that the document is part of the state of the art or is common general knowledge.

Diabetes comprises two distinct diseases, type 1 (or insulin-dependent diabetes) and type 2 (insulin-independent diabetes), both of which involve the malfunction of glucose homeostasis. Type 2 diabetes currently affects more than 400 million people in the world and this number is rising rapidly. Complications of type 2 diabetes include severe cardiovascular problems, kidney failure, peripheral neuropathy, blindness and even loss of limbs and, ultimately, death in the later stages of the disease. Type 2 diabetes is characterized by insulin resistance, and there is presently no definitive cure. Most treatments used today are focused on remedying dysfunctional insulin signalling, inhibiting glucose output from the liver or inhibiting reabsorption of glucose in the kidney but many of those treatments have several drawbacks and side effects. There is thus a great interest in identifying novel insulin-independent ways to treat type 2 diabetes.

Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, the liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (PI3K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake and glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane α-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and α_{1D} subtypes while α₂-ARs are divided into α_{2A}, α_{2B} and α_{2C}. The β-ARs are also divided into the subtypes β₁, β₂, and β₃, of which the β₂- and β₃-ARs are the major isoforms in skeletal muscle cells and adipose tissue respectively. ARs are G protein coupled receptors (GPCRs) that signal through classical secondary messengers such as cyclic adenosine monophosphate (cAMP) and phospholipase C (PLC).

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signalling increases glucose uptake is mainly via GLUT4 translocation from intracellular storage to the plasma membrane. It is known that GLUT4 translocation is induced by stimulation of the β₂-adrenergic receptors, and GLUT1 transcription and translocation are induced by stimulation of the β₃-adrenergic receptors.

β₃-Adrenergic receptors are involved in lipolysis and thermogenesis, and the use of β₃-adrenergic receptor agonists for the treatment of disorders such as obesity and type 2 diabetes has been extensively studied (see, for example, Zhu et al. Biorg. Med. Chem. Lett. (2016) 55-59). However, no effective treatments for type 2 diabetes based on this mode of action have yet been discovered.

WO 2004/110375 discloses a combination therapy based on the use of an anti-obesity agent and an anti-diabetes agent for the treatment of diabetes, wherein β₃-adrenergic receptor agonists are classified amongst the agents as anti-obesity agents for use in the co-therapy.

Various publications have suggested the use of either β₂- or β₃-adrenergic receptor agonists separately in the treatment of diabetes. However, there has been no teaching or suggestion of such a treatment based on the effect of using such agents in combination.

### DESCRIPTION OF THE INVENTION

We have now surprisingly found that the combination of a β₂-adrenergic receptor agonist and β₃-adrenergic receptor agonist represents a promising strategy for the treatment of type 2 diabetes.

Pharmaceutical Formulations

In a first aspect of the invention, there is provided a pharmaceutical formulation comprising:

(a) a β₂-adrenergic receptor agonist, or a pharmaceutically acceptable salt thereof; and

(b) a β₃-adrenergic receptor agonist, or a pharmaceutically acceptable salt thereof,

and optionally one or more pharmaceutically acceptable excipient,

which formulations may be referred to hereinafter as the “formulations of the invention”.

Unless indicated otherwise, all technical and scientific terms used herein have the same meaning as commonly understood by one of ordinary skill in the art to which this invention pertains.

Preferences and options for a given aspect, embodiment, feature or parameter of the invention should, unless the context indicates otherwise, be regarded as having been disclosed in combination with any and all preferences and options for all other aspects, features and parameters of the invention.

For the avoidance of doubt, the term “agonist” may be understood to indicate an agent that binds to a receptor and activates the receptor to produce a biological response. As such, the term also comprises partial agonists. Agonists (and partial agonists) may display, for example, half maximal effective concentration (EC₅₀) values of less than about 1 mM, such as less than about 100 μM, or less than about 10 μM, such as less than about 1 μM (e.g. less than about 100, about 10 or about 1 nM).

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. by rotary evaporation under reduced pressure, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds suitable for use in the formulations, and other aspects, of the invention (e.g. β₂- and β₃-adrenergic receptor agonists, such as those described herein) may exist as solids, and thus the scope of the invention includes all amorphous, crystalline and part crystalline forms thereof, and may also exist as oils. Where such compounds exist in crystalline and part crystalline forms, such forms may include solvates, which are included in the scope of the invention. The compounds may also exist in solution.

Suitable pharmaceutical formulations may be commercially available or otherwise are described in the literature, such as, Remington, The Science and Practice of Pharmacy, 19th ed., Mack Printing Company, Easton, Pa. (1995), and Martindale—The Complete Drug Reference (35^{th} Edition), and the documents referred to therein, the relevant disclosures in all of which documents are hereby incorporated by reference in their entirety. Otherwise, the preparation of suitable formulations, and in particular combined preparations including both a β₂- and a β₃-adrenergic receptor agonist, or pharmaceutically acceptable salts thereof, may be achieved by the skilled person using routine techniques.

References to pharmaceutically acceptable excipient(s) may be understood to include pharmaceutically acceptable, diluents, carriers and/or adjuvants, as known to those skilled in the art.

β₂-Adrenergic receptor agonists that may be employed in the various aspects of the invention include those described in WO 2004/071388, EP 0 272 976, FR 2647310, DE 2 157 040, DE 2212600, DE 2015573, ZA 6705591, DE 2128258, WO 91/09596, GB 1 199 630, DE 4209989, BE 611502, NL 7804582, EP 0 043 807, WO 2008/022038, DE 2413102, U.S. Pat. No. 2,308,232, BE 823841, BE 660244, WO 2000/075114, WO 2005/102350, WO 2005/110990, JP 56055355, AT 285583, U.S. Pat. Nos. 4,223,137, 3,056,836, FR 1324914, DE 638650, DD 45721, U.S. Pat. No. 3,801,631, DE 2259282, DE 2300614, EP 0 290 122, US 2004/0266867, US 2010/0022658, US 2010/0022659, DE 2157040, GB 2133986, WO 2006/122788, Woo et al., Molecular Pharmacology, (2009), 75(1) 158-165, Baur et al., J. Med. Chem., (2010), 53(9), 3675-3684, Kaiser et al., J. Med. Chem. (1974), 17(1) 49-57, Baker et al., J. Pharmacology and Experimental Therapeutics, (2006), 319(1), 439-446 and Engelhardt et al., Arzneimittel-Forschung, (1972), 22(5), 869-76, the relevant disclosures of which (e.g. the compounds described therein) are hereby incorporated by reference in their entirety.

In certain embodiments the invention, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672, trantinterol, clenproperol, clenpenterol, brombuterol, ractopamine and abediterol, and pharmaceutically acceptable salts thereof.

In further certain embodiments, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672 and trantinterol, and pharmaceutically acceptable salts thereof.

In particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol, trantinterol and abediterol, and pharmaceutically acceptable salts thereof.

In further particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol and trantinterol, and pharmaceutically acceptable salts thereof.

In more particular embodiments, the β₂-adrenergic receptor agonist is selected from the group consisting of formoterol, arformoterol, salmeterol, (R)-salmeterol, clenbuterol, (R)-clenbuterol, bamethane, (R)-bamethane, trantinterol and abediterol, and pharmaceutically acceptable salts thereof.

In yet more particular embodiments, the β₂-adrenergic receptor agonist is selected from the group consisting of formoterol, arformoterol, salmeterol, (R)-salmeterol, clenbuterol, (R)-clenbuterol, bamethane, (R)-bamethane and trantinterol, and pharmaceutically acceptable salts thereof.

For the avoidance of doubt, the structures of bamethane (CAS: 3703-79-5) and (R)-bamethane (CAS: 912804-58-1) are shown below.

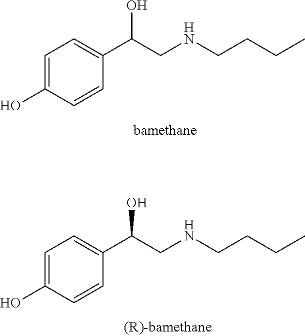

For the avoidance of doubt, in the case of a discrepancy between the name of the compound and the structure drawn in this specification, the structure should prevail.

In further embodiments, the β₂-adrenergic receptor agonist is a long-acting β₂-adrenergic receptor agonist (LABA) or an ultra-long-acting β₂-adrenergic receptor agonist (ultra-LABA), or a pharmaceutically acceptable salt thereof.

The skilled person will understand that LABAs are β₂-adrenergic receptor agonists with a duration of action of about 12 hours, and that ultra-LABAs have a duration of action of about 24 hours. Examples of LABAs include formeterol, arformeterol, salmeterol, clenbuterol, tulobuterol and bambuterol, and pharmaceutically acceptable salts thereof. Examples of ultra-LABAs include vilanterol, indacaterol, olodaterol, carmoterol and abediterol, and pharmaceutically acceptable salts thereof.

In alternative embodiments, the β₂-adrenergic receptor agonist is a short-acting β₂-adrenergic receptor agonist (SABA). In such embodiments, the SABA may be formulated as a sustained-release dosage form (i.e. provided in a pharmaceutical formulation composed of materials designed to provide sustained release of the active ingredient, as known to those skilled in the art).

The skilled person will understand that SABAs are β₂-adrenergic receptor agonists with a fast onset of effect (e.g. 20 minutes or less) and a duration of action of from about four to about six hours. Examples of SABAs include salbutamol, ritodrine, colterol, hexaprenaline and isoxsuprine, and pharmaceutically acceptable salts thereof.

In yet more particular embodiments, the β₂-adrenergic receptor agonist is clenbuterol, or a pharmaceutically acceptable salt thereof.

β₃-Adrenergic receptor agonists that may be employed in the various aspects of the invention include those described in EP 0 023 385, WO 99/20607, EP 0 303 546, EP 0 436 435, WO 99/65877, WO 2009/124166, WO 2009/124167, WO 2011/025960, WO 98/32753, EP 1 095 932, U.S. Pat. No. 5,061,727, DE 2700193, DE 2819458, EP 0 611 003, WO 96/04234, WO 98/22480, U.S. Pat. No. 4,927,836, WO 93/15041, U.S. Pat. No. 5,705,515, WO 01/74782, WO 02/232897, EP 0 659 737, EP 0 357 956, WO 97/25311, US 2003/0018061, U.S. Pat. No. 4,743,604, Edmondson et al., J. Med. Chem. (2016), 59, 609, Zhu et al., Biorg. Med. Chem. Lett. (2016), 26, 55, Mathwink et al., J. Med. Chem. (2000), 43, 3832, Fisher et al., Biorg. Med. Chem. Lett. (1996) 6, 2253, Parmee et al., Biorg. Med. Chem. Lett. (1998), 8, 1107, Sennit et al., J. Pharmacol. Exp. Ther. (1998), 285, 1084-1095 and Gavai et al., Biorg. Med. Chem. Kett. (2001) 9, 3041, the relevant disclosures of which (e.g. the compounds described therein) are hereby incorporated by reference in their entirety.

In certain embodiments of the invention, the β₃-adrenergic receptor agonist is selected from the group consisting of BRL-37344, BRL-35135, mirabegron, amirabegron, SR59104A, SR59119A, solabegron, vibegron, CAS: 1269433-49-9, CAS: 1269433-05-7, MK-0634, ritobegron, BMS-187257, CL 316,243, CGP 12177, L-755,507, L-742,791, L-750,355, L-749,372, SB-226552, SB-251023, ICI-D 7114, FR 149175, Ro40-2148, CAS: 769118-12-9, rafabegron, BMS-196085, trecadrine, SB-418790 and CAS: 99151-51-6, and pharmaceutically acceptable salts thereof.

In particular embodiments, the β₃-adrenergic receptor agonist is selected from the group consisting of BRL-37344, BRL-35135, mirabegron, amirabegron, solabegron, vibegron, CAS: 1269433-49-9, CAS: 1269433-05-7 and ritobegron, and pharmaceutically acceptable salts thereof.

In further particular embodiments, the β₃-adrenergic receptor agonist is selected from the group consisting of mirabegron, vibegron, CAS: 1269433-49-9 and CAS: 1269433-05-7, and pharmaceutically acceptable salts thereof.

In yet more particular embodiments, the β₃-adrenergic receptor agonist is mirabegron, or a pharmaceutically acceptable salt thereof.

In certain embodiments of the invention, the β₂-adrenergic receptor agonist is clenbuterol, or a pharmaceutically acceptable salt thereof; and/or (e.g. and) the β₃-adrenergic receptor agonist is mirabegron, or a pharmaceutically acceptable salt thereof.

For the avoidance of doubt, the international nonpropriety name (INN) or developmental drug code (e.g. BRL-37344) for a compound generally indicates the stereochemical configuration of the compound, or a particular mixture of stereosiomers (e.g. a racemate). Within the scope of the present invention, where relevant and unless context indicates otherwise (for example where both the racemate and a single stereoisomer are explicitly named), such names may also be considered to encompass separate stereoisomers that display the relevant biological activity, and which have not presently been assigned an alternative INN or developmental drug code.

In particular embodiments, the INN or developmental drug code should be understood to represent the compound to which the relevant name or code has been assigned only.

Where no INN or developmental drug code is available for a compound, the compound may be identified by its Chemical Abstracts Service Registry Number (CAS number). As referred to herein, the indication “CAS: XXXXXX-XX-X” (wherein the number of figures in the first group may vary) is used to identify such compounds. Where relevant and unless context indicates otherwise, the CAS number for a compound may also be considered to encompass other stereoisomers, or mixtures thereof, that display the relevant biological activity, and which have not presently been assigned alternative CAS numbers (as described above for INNs and developmental drug codes).

In particular embodiments, the CAS number should be understood to represent the compound to which the relevant name or code has been assigned only.

For the avoidance of doubt, the compounds named as CAS: 1269433-49-9, CAS: 1269433-05-7, CAS: 769118-12-9 and CAS: 99151-51-6 will be understood to have the structures shown below.

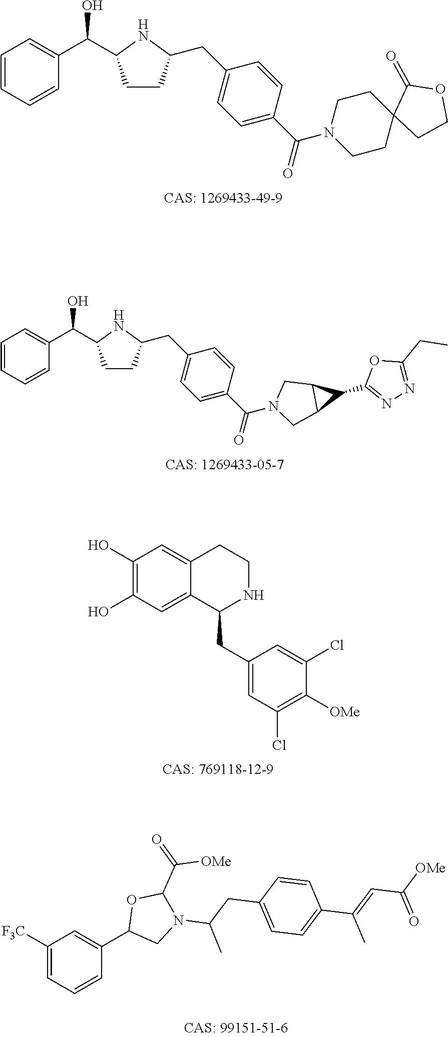 [SMILES: O=C(c1ccc(C[C@@H]2CC[C@H]([C@H](O)c3ccccc3)N2)cc1)N1CCC2(CCOC2=O)CC1 ; CCc1nnc([C@H]2[C@@H]3CN(C(=O)c4ccc(C[C@@H]5CC[C@H]([C@H](O)c6ccccc6)N5)cc4)C[C@@H]32)o1 ; COc1c(Cl)cc(C[C@@H]2NCCc3cc(O)c(O)cc32)cc1Cl ; COC(=O)/C=C(\C)c1ccc(CC(C)N2CC(c3cccc(C(F)(F)F)c3)OC2C(=O)OC)cc1]

The name CAS: 99151-51-6 designates the racemic mixture (as drawn). However, as described above, this name may, if relevant and unless context indicates otherwise, be understood to also include the single enantiomers; in particular, the (R)-enantiomer as described in U.S. Pat. No. 4,743,604, which is not presently identified by a CAS number. If it is necessary to refer to the (R)-enantiomer specifically, this compound may be referred to herein as (R)-CAS: 99151-51-6, which, for the avoidance of doubt, has the structure shown below.

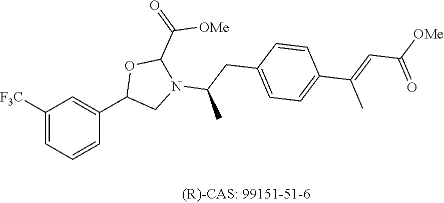 [SMILES: COC(=O)/C=C(\C)c1ccc(C[C@@H](C)N2CC(c3cccc(C(F)(F)F)c3)OC2C(=O)OC)cc1]

The present invention also embraces isotopically-labelled compounds, which are identical to the β₂- and β₃-receptor agonists recited herein but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the invention also encompasses deuterated compounds, i.e. in which one or more hydrogen atoms are replaced by the hydrogen isotope deuterium.

The β₂- and β₃-adrenergic receptor agonists employed in the formulations of the invention may typically be separate compounds, i.e. two distinct compounds, where each (at least primarily) has selective activity for a single receptor subtype. However, in certain instances, the β₂- and β₃-adrenergic receptor agonists may also take the form of a single compound displaying agonistic activity for both the β₂- and β₃-adrenergic receptor subtypes.

Thus, in certain embodiments, the formulations of the invention comprise a compound that is a β₂-adrenergic receptor agonist, or pharmaceutically acceptable salt thereof, and another (i.e. a separate and chemically different) compound that is a β₃-adrenergic receptor agonist, or a pharmaceutically acceptable salt thereof.

Kits-of-Parts

In a second aspect of the invention, there is provided a kit-of-parts comprising components:

(A) a pharmaceutical formulation comprising a β₂-adrenergic receptor agonist as defined hereinabove (i.e. in the first aspect of the invention), or a pharmaceutically acceptable salt thereof, optionally in admixture with a pharmaceutically acceptable excipient; and

(B) a pharmaceutical formulation comprising a β₃-adrenergic receptor agonist as defined hereinabove, or a pharmaceutically acceptable salt thereof, optionally in admixture with a pharmaceutically acceptable excipient, which components (A) and (B) are each provided in a form that is suitable for administration in conjunction with the other.

In an alternative second aspect of the invention, there is provided a kit-of-parts comprising:

(I) one of components (A) or (B) as defined hereinabove; and

(II) instructions to use that component in conjunction with the other of the two components.

The kits-of-parts described herein may comprise more than one formulation including an appropriate quantity/dose of a β₂-adrenergic receptor agonist, or pharmaceutically acceptable salt thereof, and/or more than one formulation including an appropriate quantity/dose of β₃-adrenergic receptor agonist, or pharmaceutically acceptable salt thereof, in order to provide for repeat dosing. If more than one formulation (comprising either active compound) is present, such formulations may be the same, or may be different in terms of the dose of either compound, chemical composition(s) and/or physical form(s).

With respect to the kits-of-parts as described herein, by “administration in conjunction with” (and similarly “administered in conjunction with”) we include that respective formulations comprising a β₂-adrenergic receptor agonist, or pharmaceutically acceptable salt thereof, and a β₃-adrenergic receptor agonist, or pharmaceutically acceptable salt thereof, are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients (i.e. a β₂-adrenergic receptor agonist, or pharmaceutically acceptable salt thereof, and a β₃-adrenergic receptor agonist, or pharmaceutically acceptable salt thereof) are administered (optionally repeatedly) either together, or sufficiently closely in time, to enable a beneficial effect for the patient, that is greater, over the course of the treatment of the relevant condition, than if either a formulation comprising a β₂-adrenergic receptor agonist, or pharmaceutically acceptable salt thereof, or a formulation comprising a β₃-adrenergic receptor agonist, or pharmaceutically acceptable salt thereof, are administered (optionally repeatedly) alone, in the absence of the other component, over the same course of treatment. Determination of whether a combination provides a greater beneficial effect in respect of, and over the course, of treatment of a particular condition will depend upon the condition to be treated or prevented, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” include that individual doses of a β₂-adrenergic receptor agonist, or pharmaceutically acceptable salt thereof, and a β₃-adrenergic receptor agonist, or pharmaceutically acceptable salt thereof, are administered within 48 hours (e.g. within 24 hours, 12 hours, 6 hours, 3 hours, 2 hours, 1 hour, 45 minutes, 30 minutes, 20 minutes or 10 minutes) of each other.

New Medical Uses and Methods of Medical Treatment

In a third aspect of the invention, there is provided a pharmaceutical formulation or kit-of-parts as defined hereinabove (i.e. as defined in the first and second aspects of the invention) for use in the treatment of type 2 diabetes.

In an alternative third aspect of the invention, there is provided the use of pharmaceutical formulation or kit-of-parts as defined hereinabove for the manufacture of a medicament for the treatment of type 2 diabetes.

In a further alternative third aspect of the invention, there is provided a method of treating type 2 diabetes, which method comprises administering a therapeutically effective amount of a pharmaceutical formulation, or a therapeutically effective amount (of the relevant active ingredient(s)) obtained from a kit-of-parts as defined hereinabove, to a patient in need of such treatment.

The skilled person will understand that references to “a therapeutically effective amount obtained from a kit-of-parts” indicates a therapeutically effective amount of both active ingredients (e.g. a β₂-adrenergic receptor agonist as defined hereinabove, or a pharmaceutically acceptable salt thereof, and a therapeutically effective amount of a β₃-adrenergic receptor agonist as defined hereinabove, or a pharmaceutically acceptable salt thereof), optionally in the form of a pharmaceutical formulation, wherein one or both of the active ingredients are obtained from a kit-of-parts as defined hereinabove. For example, a therapeutically effective amount of component (A) or (B), as defined hereinabove, obtained from a kit-of-parts.

In a fourth aspect of the invention, there is provided a method of treating type 2 diabetes, which method comprises the administration of a therapeutically effective amount of a β₂-adrenergic receptor agonist as defined hereinabove, or a pharmaceutically acceptable salt thereof, and (i.e. in combination with) a therapeutically effective amount of a β₃-adrenergic receptor agonist as defined hereinabove, or a pharmaceutically acceptable salt thereof, to a patient in need thereof.

In a fifth aspect of the invention, there is provided a β₂-adrenergic receptor agonist as defined hereinabove, or a pharmaceutically acceptable salt thereof, for use in the treatment of type 2 diabetes, wherein the treatment comprises administration in combination with a β₃-adrenergic receptor agonist as defined hereinabove, or a pharmaceutically acceptable salt thereof.

In an alternative fifth aspect of the invention, there is provided the use of a β₂-adrenergic receptor agonist as defined hereinabove, or a pharmaceutically acceptable salt thereof, for the manufacture of a medicament for the treatment of type 2 diabetes, wherein the treatment comprises administration in combination with a β₃-adrenergic receptor agonist as defined hereinabove, or a pharmaceutically acceptable salt thereof.

In a sixth aspect of the invention, there is provided a β₃-adrenergic receptor agonist as defined hereinabove, or a pharmaceutically acceptable salt thereof, for use in the treatment of type 2 diabetes, wherein the treatment comprises administration in combination with a β₂-adrenergic receptor agonist as defined hereinabove, or a pharmaceutically acceptable salt thereof.

In an alternative sixth aspect of the invention, there is provided the use of a β₃-adrenergic receptor agonist as defined hereinabove, or a pharmaceutically acceptable salt thereof, for the manufacture of a medicament for the treatment of type 2 diabetes, wherein the treatment comprises administration in combination with a β₂-adrenergic receptor agonist as defined hereinabove, or a pharmaceutically acceptable salt thereof.

The skilled person will understand that references to the “treatment of” a particular condition (and similarly “treating”) take their normal meanings in the field of medicine. In particular, the terms may refer to achieving a reduction in the severity of one or more clinical symptom associated with the condition. For example, in the case of type 2 diabetes, the term may refer to achieving a reduction of blood glucose levels.

As used herein, references to patients will refer to a living subject being treated, including mammalian (e.g. human) patients. Thus, in particular embodiments of the relevant aspects of the invention (e.g. the third to sixth aspects of the invention), the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

In particular embodiments of the third to sixth aspects of the invention, the disorder is type 2 diabetes selected from the list consisting of maturity-onset diabetes in the young (MODY), ketosis-prone diabetes in adults, latent autoimmune diabetes of adults (LADA), and gestational diabetes.

For the avoidance of doubt, the methods, pharmaceutical formulations for use, kits-of-parts for use, compounds for use and uses as described for the third to sixth aspects of the invention may collectively be referred to as “treatments of the third to sixth aspects of the invention”.

The skilled person will understand that in the course of the treatments of the third to sixth aspects of the invention, the β₂-adrenergic receptor agonist, or pharmaceutically acceptable salt thereof, and the β₃-adrenergic receptor agonist, or pharmaceutically acceptable salt thereof, may be administered, sequentially, separately and/or simultaneously, over the course of treatment of the relevant condition (i.e. administered in conjunction with each other, as defined for the kits-of-parts of the second aspect of the invention).

The skilled person will understand that treatments of the third to sixth aspects of the invention may further comprise (i.e. be combined with) further (i.e. additional/other) treatment(s) for the same condition. In particular, treatment may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

In particular, the treatments of the third to sixth aspects of the invention may be performed in combination with (e.g. in a patient who is also being treated with) one or more (e.g. one) additional compounds (i.e. therapeutic agents) that:

(i) are capable of reducing blood sugar levels; and/or

(ii) are insulin sensitizers; and/or

(iii) enhance insulin release,

which agents will be readily identified by those skilled in the art; in particular, including such therapeutic agents that are commercially available (e.g. agents that the subject of a marketing authorization in one or more territory, such as a European or US marketing authorization). Such agents may also be administered in conjunction, as defined hereinabove, with the treatments of the third to sixth aspects of the invention.

The skilled person will understand that references to therapeutic agents capable of reducing blood glucose levels may refer to compounds capable of reducing glucose levels in blood to at least 10 mmol/mL (such as at least 7.5 mmol/mL or at least 6 mmol/mL, e.g. to 5.5 mmol/mL) when compared to the blood glucose levels prior to treatment with the relevant compound.

The skilled person will understand that β₂- and β₃-adrenergic receptor agonists, or pharmaceutically acceptable salts thereof, as defined herein (i.e. the active compounds associated with the various aspects of the invention) will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical formulations as described herein will include compositions in the form of tablets, capsules or elixirs for oral administration, suppositories for rectal administration, sterile solutions or suspensions for parenteral or intramuscular administration, and the like.

Thus, in particular embodiments of the first and second aspects of the invention, the pharmaceutical formulation(s) is/are provided in a pharmaceutically acceptable dosage form, including tablets or capsules, liquid forms to be taken orally or by injection, suppositories, creams, gels, foams, inhalants (e.g. to be applied intranasally). For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that the β₂-adrenergic receptor agonist, or pharmaceutically acceptable salt thereof, and β₃-adrenergic receptor agonist, or pharmaceutically acceptable salt thereof, of the first to sixth aspects of the invention may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 mg/kg of body weight per day (mg/kg/day) to about 200 mg/kg/day, preferably about 0.01 to about 10 mg/kg/day, and more preferably about 0.1 to about 5.0 mg/kg/day. For example, when administered orally, treatment with such compounds may comprise administration of a formulations typically containing between about 0.01 mg to about 2000 mg, for example between about 0.1 mg to about 500 mg, or between 1 mg to about 100 mg, of the active ingredient(s). When admixture intravenously, the most preferred doses will range from about 0.001 to about 10 mg/kg/hour during constant rate infusion. Advantageously, treatment may comprise administration of such compounds and compositions in a single daily dose, or the total daily dosage may be administered in divided doses of two, three or four times daily (with reference to the doses described herein).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; however, there can, of course, be individual instances where higher or lower dosage ranges are merited, and such are included within the scope of this invention.

As described herein above, the skilled person will understand that the treatments of the third to sixth aspects of the invention may further comprise (i.e. be combined with) further (i.e. additional/other) treatment(s) for the same condition. In particular, treatment may be combined with other means for the treatment of type 2 diabetes, such as treatment with one or more other therapeutic agent that is useful in the treatment of type 2 diabetes.

In particular embodiments of the first and second aspects of the invention, the pharmaceutical formulations or kits-of-parts may further comprise one or more additional (i.e. other) therapeutic agent.

In more particular embodiments, the one or more additional therapeutic agent is an agent for the treatment of type 2 diabetes as known to those skilled in the art, such as metformin, sulfonylureas (e.g. carbutamide, acetohexamide, chlorpropamide, tolbutamide. glipizide (glucotrol), gliclazide, glibenclamide, glyburide (Micronase), glibornuride, gliquidone, glisoxepide, glyclopyramide, glimepiride (Amaryl), glimiprime, JB253 or JB558), thiazolidinediones (e.g. pioglitazone, rosiglitazone (Avandia), lobeglitazone (Duvie) and troglitazone (Rezulin)), dipeptidyl peptidase-4 inhibitors (e.g. sitagliptin, vildagliptin, saxagliptin, linagliptin, anagliptin, teneligliptin, alogliptin, trelagliptin, gemigliptin, dutogliptin and omarigliptin), SGLT2 inhibitors (e.g. dapagliflozin, empagliflozin, canagliflozin, ipragliflozin, tofogliflozin, sergliflozin etabonate, remogliflozin etabonate, and ertugliflozin), and glucagon-like peptide-1 (GLP-1) analogues.

Preparation of Formulations and Kits-of-Parts

Pharmaceutical formulations and kits-of-parts as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a β₂-adrenergic receptor agonist, or pharmaceutically acceptable salt thereof, and a β₃-adrenergic receptor agonist, or pharmaceutically acceptable salt thereof, as hereinbefore defined, with one or more pharmaceutically-acceptable excipients (e.g. an adjuvant, diluent and/or carrier).

In further aspects of the invention, there is provided a process for the preparation of a kit-of-parts as hereinbefore defined, which process comprises bringing into association a β₂-adrenergic receptor agonist, or pharmaceutically acceptable salt thereof, or a β₃-adrenergic receptor agonist, or pharmaceutically acceptable salt thereof, as hereinbefore defined, or a pharmaceutically acceptable salt thereof with the other therapeutic agent that is useful in the treatment of cancer, and at least one pharmaceutically-acceptable excipient.

There is further provided a method of making a kit-of-parts as defined hereinbefore, which method comprises bringing component (A) into association with component (B), thus rendering the two components suitable for administration in conjunction with each other.

As used herein, references to bringing into association will mean that the two components are rendered suitable for administration in conjunction with each other.

Thus, in relation to the process for the preparation of a kit of parts as hereinbefore defined, by bringing the two components “into association with” each other, it is contemplated that the two components of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Wherever the word “about” is employed herein (for example, in the context of doses of active ingredients) it will be appreciated that such variables are approximate and as such may vary by ±10%, for example ±5% and preferably ±2% (e.g. ±1%) from the numbers specified herein.

The pharmaceutical formulations, kits-of-parts, methods, uses and compounds for use described herein may have the advantage that, in the treatment of the conditions mentioned hereinbefore, they may be more convenient for the physician and/or patient than, be more efficacious than, be less toxic than, have a broader range of activity than, be more potent than, produce fewer side effects than, or may have other useful pharmacological properties over, similar methods (treatments) known in the prior art for use in the treatment of type 2 diabetes or otherwise. In particular, it is believed by the present inventors that combinations of active ingredients as described herein have particular efficacy in the treatment of type 2 diabetes, which would not be reasonably expected by those skilled in the art.

### EXAMPLES

The present invention is illustrated by way of the following examples, which are not intended to be limiting.

### Example 1

A glucose tolerance test (GTT) was performed in mice fed on a high fat diet and pre-treated with intraperitoneal injections of clenbuterol (1 mg/kg), mirabegron (1 mg/kg) or the combination of clenbuterol+mirabegron for 4 consecutive days.

For the GTT, mice were fasted for 6 hours and then injected with glucose (2.5 mg/kg lean body mass). Blood glucose concentrations were determined with a glucometer using blood taken from cut tail tips.

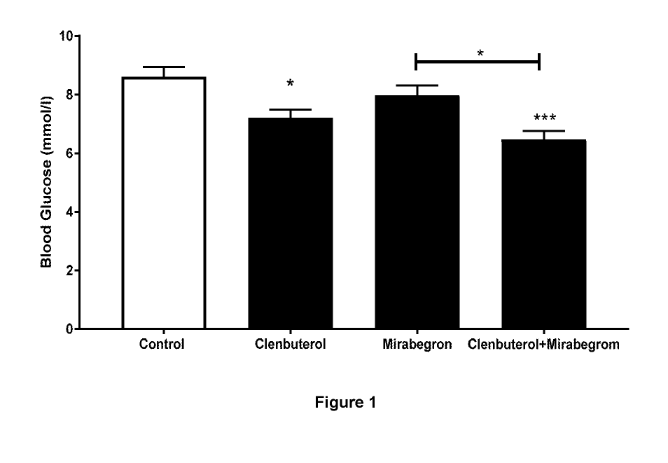

The results obtained are shown in FIG. 1. Values are means±SEM of 6 mice in each group. *P<0.05 vs. control at a specific time point of 120 min.

1. A pharmaceutical formulation comprising:
(a) a β₂-adrenergic receptor agonist, or a pharmaceutically acceptable salt thereof; and
(b) a β₃-adrenergic receptor agonist, or a pharmaceutically acceptable salt thereof, and optionally one or more pharmaceutically acceptable excipient.

2. The pharmaceutical formulation as claimed in claim 1, wherein the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, clencyclohexerol, tulobuterol, BRL-47672, trantinterol, clenproperol, clenpenterol, brombuterol, ractopamine and abediterol, and pharmaceutically acceptable salts thereof.

3. The pharmaceutical formulation as claimed in claim 1, wherein the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, clencyclohexerol, tulobuterol, trantinterol and abediterol, and pharmaceutically acceptable salts thereof.

4. The pharmaceutical formulation as claimed in claim 1, wherein the β₂-adrenergic receptor agonist is selected from the group consisting of formoterol, arformoterol, salmeterol, (R)-salmeterol, clenbuterol, (R)-clenbuterol, bamethane, trantinterol and abediterol, and pharmaceutically acceptable salts thereof.

5. The pharmaceutical formulation as claimed in claim 1, wherein the β₂-adrenergic receptor agonist is a long-acting β₂-adrenergic receptor agonist or an ultra-long-acting β₂-adrenergic receptor agonist, or a pharmaceutically acceptable salt thereof.

6. The pharmaceutical formulation as claimed in claim 1, wherein the β₂-adrenergic receptor agonist is clenbuterol, or a pharmaceutically acceptable salt thereof.

7. The pharmaceutical formulation as claimed in claim 1, wherein the β₃-adrenergic receptor agonist is selected from the group consisting of BRL-37344, BRL-35135, mirabegron, amirabegron, SR59104A, SR59119A, solabegron, vibegron, CAS: 1269433-49-9, CAS: 1269433-05-7, MK-0634, ritobegron, BMS-187257, CL 316,243, CGP 12177, L-755,507, L-742,791, L-750,355, L-749,372, SB-226552, SB-251023, ICI-D 7114, FR 149175, Ro40-2148, CAS: 769118-12-9, rafabegron, BMS-196085, trecadrine, SB-418790 and CAS: 99151-51-6, and pharmaceutically acceptable salts thereof.

8. The pharmaceutical formulation as claimed in claim 1, wherein the β₃-adrenergic receptor agonist is selected from the group consisting of BRL-37344, BRL-35135, mirabegron, amirabegron, solabegron, vibegron, CAS: 1269433-49-9, CAS: 1269433-05-7 and ritobegron, and pharmaceutically acceptable salts thereof.

9. The pharmaceutical formulation as claimed in claim 1, wherein the β₃-adrenergic receptor agonist is selected from the group consisting of mirabegron, vibegron, CAS: 1269433-49-9 and CAS: 1269433-05-7, and pharmaceutically acceptable salts thereof.

10. The pharmaceutical formulation as claimed in claim 1, wherein the β₃-adrenergic receptor agonist is mirabegron, or a pharmaceutically acceptable salt thereof.

11. The pharmaceutical formulation as claimed in claim 1, wherein the β₂-adrenergic receptor agonist is clenbuterol, or a pharmaceutically acceptable salt thereof, and the β₃-adrenergic receptor agonist is mirabegron, or a pharmaceutically acceptable salt thereof.

12. A kit-of-parts comprising components:
(A) a pharmaceutical formulation comprising a β₂-adrenergic receptor agonist as defined in claim 1, or a pharmaceutically acceptable salt thereof, optionally in admixture with a pharmaceutically acceptable excipient; and
(B) a pharmaceutical formulation comprising a β₃-adrenergic receptor agonist as defined in claim 1, or a pharmaceutically acceptable salt thereof, optionally in admixture with a pharmaceutically acceptable excipient,
which components (A) and (B) are each provided in a form that is suitable for administration in conjunction with the other.

13. A kit-of-parts comprising:
(I) one of components (A) or (B) as defined in claim 12; and
(II) instructions to use that component in conjunction with the other of the two components.

14-15. (canceled)

16. A method of treating type 2 diabetes, which method comprises administering a therapeutically effective amount of the pharmaceutical formulation as claimed in claim 1 to a patient in need of such treatment.

17. A method of treating type 2 diabetes, which method comprises the administration of a therapeutically effective amount of a β₂-adrenergic receptor agonist as defined in claim 1, or a pharmaceutically acceptable salt thereof, and a therapeutically effective amount of a β₃-adrenergic receptor agonist as defined in claim 1, or a pharmaceutically acceptable salt thereof, to a patient in need thereof.

18-21. (canceled)
